(19) United States
(12) Patent Application Publication
(10) Pub. No.: US 2015/0105666 A1
(43) Pub. Date: Apr. 16, 2015

(54) NARROW BAND FEATURE EXTRACTION FROM CARDIAC SIGNALS

(71) Applicant: OBS MEDICAL LIMITED, Oxfordshire (GB)

(72) Inventor: Iain Guy David Strachan, Oxfordshire (GB)

(21) Appl. No.: 14/404,024

(22) PCT Filed: May 28, 2013
(86) PCT No.: PCT/GB2013/051408

§ 371 Date: Nov. 26, 2014

(30) Foreign Application Priority Data
May 28, 2012 (GB) 1209412.4

(51) Int. Cl. A61B 5/04; A61B 5/1455; A61B 5/0456; A61B 5/024; A61B 5/0245

(52) U.S. Cl. CPC A61B 5/04012; A61B 5/02416; A61B 5/0245; A61B 5/0456; A61B 5/1455
USPC 600/473; 600/508; 600/479

(57) ABSTRACT

A PPG or other cardiac signal is analysed by calculating its amplitude in a narrow frequency range around the estimated heart rate or a harmonic of the heart rate. Cardiac signals from a patient in different states, e.g., exercise and non-exercise or limb lowered and limb raised can be analysed and the amplitude in the narrow range compared to determine various vascular conditions such as peripheral arterial disease.

The present invention relates to a method of and apparatus for processing a cardiac signal from a human or animal subject to allow detection of some aspect of the condition of the subject. More particularly, the method involves analysing the features of a narrow frequency band of the cardiac signal as a way of improving the robustness of the result.

It is well known to obtain cardiac signals from human or animal subjects, for example electrocardiograms (ECG) or photoplethysmograms (PPG) and to examine or analyse these signals to determine some aspect of the condition of the subject. In common with many signals from human or animal subjects, however, the quality of the signals can vary greatly depending on accuracy and security of sensor positioning, and the signals are inherently noisy. There is a widespread need and interest, therefore, in processing such signals to allow greater accuracy and robustness in the conclusions to be drawn from them.

One example of analysis of cardiac signals is in the detection of Peripheral Arterial Disease (PAD) in which PPG signals are obtained from two pulse oximeter sensors, one mounted on the toe and one on the foot. Each sensor provides two separate PPG signals, one at infra red and one at red frequencies. Conventionally, two 30 second segments of data are collected from a supine subject, one with the leg lowered and one with the leg raised above the level of the heart. For healthy subjects, there is an increase in the amplitude of the PPG waveform when the leg is raised because the heart has to work harder to pump blood above the level of the heart. In a diseased patient, however, the heart is already working harder due to the occlusion in the arteries in the leg, and no amplitude increase (or sometimes an amplitude decrease) is observed. Typically, to classify the patient as diseased or not, the root mean square (RMS) amplitude over the 30 second period is calculated for each of the eight signals (IR and red signals for each of the toe and foot sensors in each of the lowered and raised position), and a weighted average is calculated of all of them with the weight coefficients being set to distinguish between diseased and normal patients by means of multiple linear regression of a set of empirical training data. However, the waveforms collected are often corrupted by noise which may be due to movement artefact or poor sensor placement. This noise introduces errors into the calculation of the RMS amplitude.

Another example of cardiac signal analysis is in the analysis of ECG waveforms to obtain a respiration rate. It is known that respiration causes a periodic variation in the heart rate, but again it can be difficult to separate this signal given the amount of noise and possible movement artefact.

Accordingly, one aspect of the present invention provides a method of processing a cardiac signal from a human or animal subject to detect an indication of a vascular condition, comprising the steps of:
obtaining a cardiac signal for each of two different states of the subject;
estimating the heart rate in each cardiac signal;
determining, for each cardiac signal, a value representative of the amplitude of the cardiac signal over a predetermined limited range of frequencies around the frequency corresponding to the estimated heart rate or to a harmonic of the estimated heart rate; and
comparing the determined values to detect said indication of a vascular condition.

Another aspect of the present invention provides a computer program comprising computer-executable code that when executed on a computer system causes the computer system to perform a method according to any one of the preceding claims. A further aspect of the invention provides a computer-readable medium storing a computer program according to the preceding aspect of the invention.

A yet further aspect of the invention provides an apparatus for processing a cardiac signal from a human or animal subject to detect an indication of a vascular condition, comprising:
an input section configured to receive a cardiac signal for a plurality of different states of the subject;
an estimation section configured to estimate the heart rate in each cardiac signal;
a determination section configured to determine, for each cardiac signal, a value representative of the amplitude of the cardiac signal over a predetermined limited range of frequencies around the frequency corresponding to the estimated heart rate or to a harmonic of the estimated heart rate; and
a comparison section configured to compare the determined values to detect said indication of a vascular condition

Thus, with the present invention the amplitude feature of the cardiac signal is limited to a predetermined narrow range around the estimated heart rate or a harmonic (i.e. a multiple) of the estimated heart rate. This can remove noise and artefact and thus result in a more accurate amplitude measurement in contrast with the prior art broadband approach of computing the RMS power in the whole signal. The fundamental (i.e. frequency corresponding to the heart rate) may be used, or the first or second harmonic (double or triple the frequency corresponding to the heart rate).

Preferably, the values representative of the amplitude of the cardiac signal are obtained by measuring the power in the cardiac signals over the predetermined limited range of frequencies. This can be done by computing the area under the curve of a frequency domain representation of each cardiac signal over the predetermined limited range of frequencies.

Alternatively, the cardiac signals can be transformed into the frequency domain, for example by a Fast Fourier Transform, spectral components outside the predetermined limited range of frequencies can then be easily removed, the signals converted back into the time domain and the amplitude (for example the RMS amplitude) measured.

Another alternative way of obtaining the values representative of the amplitude of the cardiac signals is to bandpass filter the cardiac signals to remove frequencies outside the predetermined limited range. Again, the predetermined limited range can be around the frequency corresponding to the heart rate, or around a harmonic of that frequency.

An advantage of determining the value representative of the amplitude over a predetermined limited range around a harmonic of the heart rate is that this can be at a frequency which is far removed from any noise or movement artefact.

The method is applicable to PPG signals, for example in the red and infra red region for detecting PAD as mentioned above, or two ECG signals.

The different states of the subject could correspond to the subject's body position being changed, or to the subject during exercise and relaxation.

In the case of detection of PAD, the two signals also come from two different parts of the subject's body, for example the foot and toe.

It should be appreciated therefore that the invention can be applied to two or more signals, from the same or different sensors.

The comparison of the two values representative of amplitude can comprise calculating the difference between them or calculating a weighted sum of the values. The result can be compared with a threshold. The values, or the result of the difference or weighted sum calculation can be compared with corresponding values from a training set which can include values from normal and abnormal subjects (e.g. diseased and not diseased).

The heart rate can be estimated by a variety of known methods, for example the detection of peaks in the cardiac signals. Preferably, a value for the confidence of the estimate of heart rate is also obtained, for example by comparing the heart rate estimate to the nearest maximum in the power spectrum of the cardiac signal. Further measures of confidence can be obtained by comparing the nearest maximum in the power spectrum with a harmonic of the estimated heart rate and also by checking the heart rate estimate against the normal heart rate range for that type of subject.

Embodiments of the invention will be further described by way of example with reference to the accompanying drawings in which:

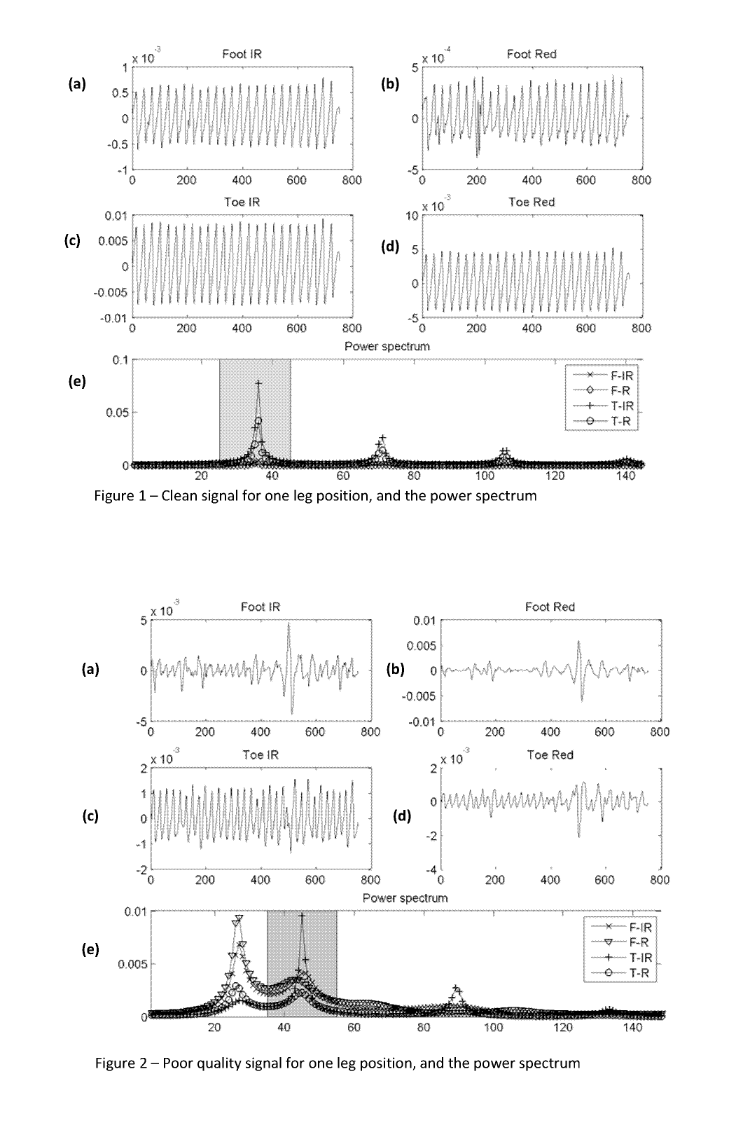

FIGS. 1(a) to (e) show example PPG signals obtained from a subject's foot and toe in the red and infra red regions, together with the corresponding power spectrum (frequency domain representation) of the four signals;

FIGS. 2(a) to (e) show example poor quality PPG signals obtained from the foot and toe of a subject in the red and infra red regions, together with the corresponding power spectrum (frequency domain representation);

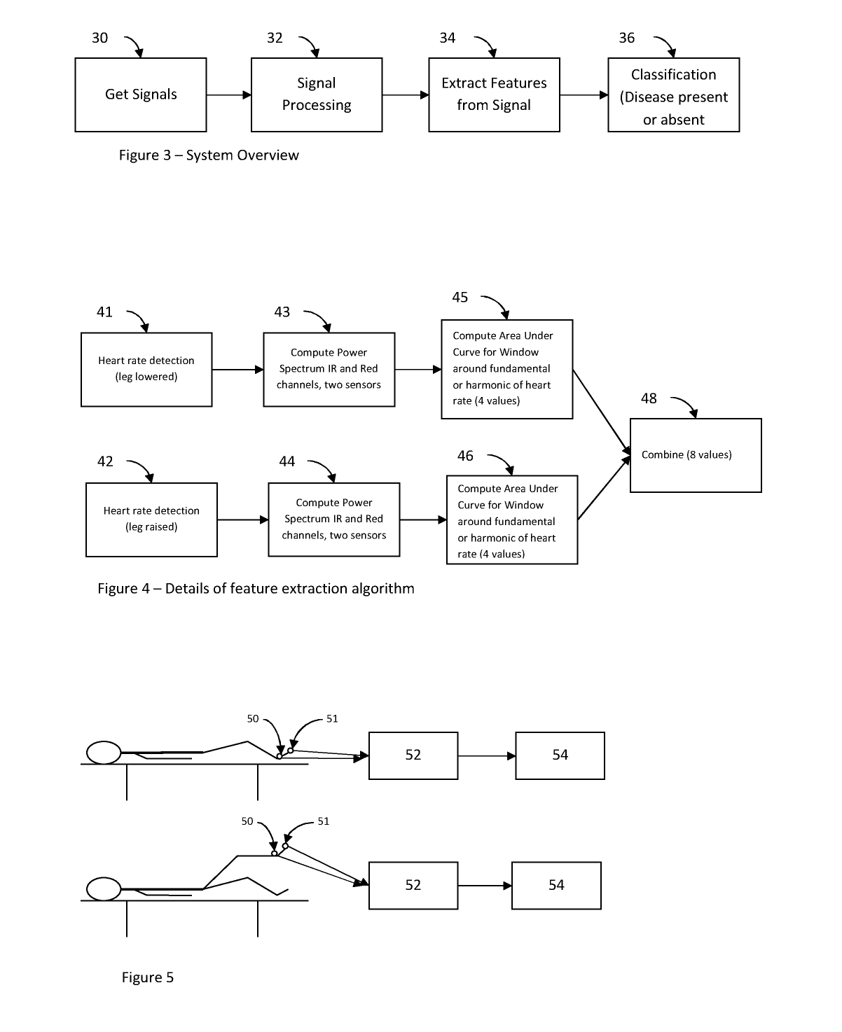

FIG. 3 schematically illustrates the process of one embodiment of the invention;

FIG. 4 schematically illustrates one embodiment of the extraction of features from a PPG signal; and

FIG. 5 schematically illustrates PPG measurements to detect Peripheral Arterial Disease in a human subject.

As illustrated in FIG. 5, one embodiment of the present invention may be used in the analysis of PPG signals used to detect Peripheral Arterial Disease. FIG. 5 schematically illustrates the way such signals are obtained, as mentioned above PPG signals in both the red and infra red region are obtained from both the foot and toe of a subject with the leg first lowered and then raised. The signals from the foot and toe sensors (50, 51) are collected by a PPG controller (52) and then output to a data processor (54) which analyses the signals as explained below and outputs the results.

FIG. 1 illustrates in FIGS. 1(a) to (d), four good quality PPG signals obtained in the infra red and red regions for the foot and the toe. All of the signals are relatively clean, apart from the foot red sensor which shows a small artefact at around 200 samples. FIG. 1(e) shows the power spectra (frequency domain representations) of the four sensors, with a narrow band highlighted around the “fundamental” frequency, which corresponds to the heart rate. The present invention, as explained below, analyses the amplitude within that narrow band, or within a similar narrow band around one of the harmonics which are visible at frequency (x-axis) values of about 75 and 105. It is necessary to use a small band around the heart rate or fundamental thereof because the heart rate varies slightly from beat to beat with the respiratory cycle (known as Respiratory Sinus Arrhythmia).

By way of comparison, FIG. 2 illustrates that poor quality signals for foot and toe PPG in the infra red and red regions with three of the four sensors showing a large spike-like artefact just after 500 samples. This appears in the power spectrum of FIG. 2(e) as a secondary peak at a lower frequency in all four power spectra. This peak influences the height of the curves at the fundamental frequency. In this case, although the illustrated narrow band around the “fundamental” frequency excludes the noise, it can be seen that using one of the harmonic peaks would result in significantly less pollution by the artefact.

FIG. 3 schematically illustrates the overview of the processing of the signals. In step 30 the signals are obtained by means of PPG sensors and controller (50, 51 and 52) and then steps 32, 34 and 36 the signals are processed, the amplitude features in the frequency band under consideration extracted, and the subject classified by means of the processor 54.

In more detail, the PPG signals are collected for 30 seconds with the leg lowered and also then with the leg raised as shown in steps 41 and 42 of FIG. 4. The heart rate is then robustly determined for the 30 second interval (that is to say a heart rate is estimated with the leg lowered and another heart rate estimated with the leg raised). This can be done by any known technique, but in this embodiment the most stable of the four wave forms is selected (either by a heuristic such as measuring the noise level, or simply choosing the most consistent channel, e.g. the toe IR, from prior observation), and performing a simple peak detection algorithm on it to locate the maxima of the signal. A typical public domain peak detection algorithm involves searching for a maximum by detecting whether the signal has fallen a fixed amount below the current “maximum”, and if so marking it as a peak. This peak detection algorithm therefore gives a number of “instantaneous heart rate” estimates in terms of the peak-to-peak times. An estimate of heart rate for the 30 second interval can be determined based on the median peak-to-peak time, excluding those whose peak-to-peak distance would correspond to an unrealistic heart rate (e.g. outside the normal range of 45 to 150 beats per minute).

A further check on the integrity of the estimate may be made by comparing the power spectrum of the cardiac signal in the region of the estimated heart rate. If the nearest maximum in the power spectrum is not within a specified tolerance of the estimated heart rate, then the data is deemed unmeasurable.

A similar check may also be applied to the maxima in the power spectrum at multiples (harmonics) of the estimated heart rate.

Once the heart rate has been robustly estimated (the heart rate will be the same for all four sensors), in steps 43 and 44 the power spectrum in the vicinity of the fundamental or a harmonic of the heart rate is computed over a narrow band of frequencies. The narrow band is defined in this embodiment as +/−10 bins of the 1024 point FFT which corresponds to a frequency range of about 0.5 Hz. This is based on the variability of the heart rate and determined empirically during the training process, and would typically be in the range 0.2 to 0.5 Hz. Having calculated the power spectrum, for example resulting in a plot as illustrated in FIG. 1(e) or FIG. 2(e), an amplitude-like feature can be computed in steps 45 and 46 by calculating the area under the curve of the plot over the narrow band. The power spectrum and amplitude calculation can be done in a variety of ways:

(a) by a Fast Fourier Transform whose length is the next power of two greater than the number of samples (in this case there are 750 samples for a 30 second interval so the length of the FFT will be 1024). The power spectrum is then computed from the absolute value of the complex-valued FFT spectrum.

(b) by the “All Poles” method. In this method the linear prediction coefficients of the waveform are computed via the Yule-Walker equations. The linear prediction coefficients obtained represent the denominator of a polynomial function in the complex domain which can be evaluated at a sequence of points on the unit circle in the complex domain. Poles corresponding to significant spectral content in the signal can be identified and their distance from the origin in the complex domain represents the amplitude. (This is a well-known technique based on the original papers: G. Udny Yule “On a Method of Investigating Periodicities in Disturbed Series, with Special Reference to Wolfer's Sunspot Numbers” Philosophical Transactions of the Royal Society of London, Ser. A, Vol. 226, (1927) 267-298; and Gilbert Walker “On Periodicity in Series of Related Terms,” Proceedings of the Royal Society of London, Ser. A, Vol. 131, (1931) 518--532. More modern explanations can be found on the web.)

The amplitude-like feature in the narrow band is computed for all eight signals (infra red and red for foot and toe with the leg raised and lowered).

The eight values thus calculated are then used in step 48 to compute an index I by applying a weighted sum according to the formula:

I=a+Σ₁₌₁ⁱ⁼⁸bᵢxᵢ

Where the constant offset a and weighting coefficients bᵢ are determined by multiple linear regression from a training set of previously-acquired PPG readings, together with an assessment of the disease/non-disease state determined by alternative diagnostic methods. The subject is classified as disease positive if the index is below a predetermined threshold, or as clear if the index is above the threshold.

An alternative way of measuring the amplitude, rather than computing the area under the curve of the power spectrum in the narrow frequency band is to calculate the root mean square (RMS) value of the cardiac signals in the time domain after having removed undesired spectral content. This can be achieved in one of two ways.

(A) The first way is to transform the cardiac signals into the frequency domain, for example by computing the 1024 point complex-valued FFT. Then all entries in the FFT outside the desired frequency window around the fundamental or selected harmonic are set to 0. The FFT is symmetric so this involves leaving non-zero data in either half of the spectrum. The signal is then converted back into the time domain by computing the inverse FFT, and the RMS value of the resultant signal for the 30 seconds can be computed. Because the resultant signal has had its spectral content limited to the narrow region around the fundamental or a harmonic, it becomes an approximate sinusoid at the heart rate or one of its multiples.

(B) Another alternative approach is to digitally bandpass filter the cardiac signals using a bandpass filter set to pass only the narrow desired range of frequencies around the fundamental or selected harmonic, and then to calculate the RMS value for 30 seconds as mentioned above.

Although the invention has been described above with reference to methods embodying the invention, the invention can also be embodied as apparatus. For example, an apparatus for processing a cardiac signal from a human or animal subject to detect an indication of a vascular condition, comprising:

an input section configured to receive a cardiac signal for a plurality of different states of the subject;

an estimation section configured to estimate the heart rate in each cardiac signal;

a determination section configured to determine, for each cardiac signal, a value representative of the amplitude of the cardiac signal over a predetermined limited range of frequencies around the frequency corresponding to the estimated heart rate or to a harmonic of the estimated heart rate; and

a comparison section configured to compare the determined values to detect said indication of a vascular condition.

Furthermore, any of the detailed method features described above with reference to the method embodiments can be embodied as apparatus sections.

It is possible to implement the apparatus sections as dedicated hard-wired electronic circuits; however the various sections do not have to be separate from each other, and could all be integrated onto a single electronic chip. Furthermore, the sections can be embodied as a combination of hardware and software, and the software can be executed by any suitable general-purpose microprocessor, such that in one embodiment the apparatus can be a conventional personal computer (PC), such as a standard desktop or laptop computer, or can be a dedicated device.

The invention can also be embodied as a computer program stored on any suitable computer-readable storage medium, such as a solid-state computer memory, a hard drive, or a removable disc-shaped medium in which information is stored magnetically, optically or magneto-optically. The computer program comprises computer-executable code that when executed on a computer system causes the computer system to perform a method embodying the invention.

1. A method of processing a cardiac signal from a human or animal subject to detect an indication of a vascular condition, comprising the steps of:
obtaining a cardiac signal for a plurality of different states of the subject;
estimating the heart rate in each cardiac signal;
determining, for each cardiac signal, a value representative of the amplitude of the cardiac signal over a predetermined limited range of frequencies around the frequency corresponding to the estimated heart rate or to a harmonic of the estimated heart rate; and
comparing the determined values to detect said indication of a vascular condition.

2. A method according to claim 1 wherein the values representative of the amplitude of the cardiac signals are obtained by measuring the power in a frequency domain representation of the cardiac signals over the predetermined limited range of frequencies.

3. A method according to claim 2 wherein the power in the frequency domain representation of the cardiac signals over the predetermined limited range of frequencies is measured by computing the area under the curve of a frequency domain representation of each signal over the predetermined limited range of frequencies.

4. A method according to claim 1 wherein the values representative of the amplitude of the cardiac signals are obtained by conversion of the cardiac signals to the frequency domain, removing spectral components outside the predetermined limited range of frequencies, converting the signals back to the time domain and measuring their amplitude.

5. A method according to claim 1 wherein the values representative of the amplitude of the cardiac signals are obtained by bandpass filtering the cardiac signals to remove frequencies outside the predetermined limited range of frequencies and measuring the amplitude of the bandpass filtered cardiac signals.

6. A method according to claim 1 wherein the cardiac signals are photoplethysmograms.

7. A method according to claim 6 wherein red and infra red photoplethysmogram signals are obtained for each of two states of the subject.

8. A method according to any claim 1 wherein the photoplethysmograms are obtained from different parts of body of the subject.

9. A method according to claim 1 wherein the step of comparing the states comprises calculating the relationship between them.

10. A method according to claim 1 wherein the step of comparing the states comprises the step of calculating a weighted sum of them.

11. A method according to claim 1 wherein the step of comparing the states comprises a step of comparison with a threshold.

12. A method according to claim 1 wherein the step of comparing the states comprises the step of comparison with values in, or obtained from, a training set of values.

13. A method according to claim 1 wherein the step of estimating the heart rate comprises detection of peaks in the cardiac signals.

14. A method according to claim 1 further comprising calculating a measure of confidence in the heart rate estimate by comparing the heart rate estimate to the nearest maximum in the power spectrum of the cardiac signals in the physiological range of heart rates.

15. A computer program comprising computer-executable code that when executed on a computer system causes the computer system to perform a method according to claim 1.

16. A computer-readable medium storing a computer program according to claim 15.

17. An apparatus for processing a cardiac signal from a human or animal subject to detect an indication of a vascular condition, comprising:
an input section configured to receive a cardiac signal for a plurality of different states of the subject;
an estimation section configured to estimate the heart rate in each cardiac signal;
a determination section configured to determine, for each cardiac signal, a value representative of the amplitude of the cardiac signal over a predetermined limited range of frequencies around the frequency corresponding to the estimated heart rate or to a harmonic of the estimated heart rate; and
a comparison section configured to compare the determined values to detect said indication of a vascular condition.
